(19) United States
(12) Patent Application Publication
(10) Pub. No.: US 2022/0316012 A1
(43) Pub. Date: Oct. 6, 2022

(54) METHOD FOR PREDICTING IMMUNOTHERAPY RESPONSE WITH CORRECTED TMB

(71) Applicant: GENINUS INC., Songpa-gu, Seoul (KR)

(72) Inventors: Woong-Yang PARK, Gangnam-gu, Seoul (KR); Se-Hoon LEE, Gangnam-gu, Seoul (KR); Joon Ho SHIM, Gangnam-gu, Seoul (KR)

(73) Assignee: GENINUS INC., Songpa-gu, Seoul (KR)

(21) Appl. No.: 16/972,811

(22) PCT Filed: Oct. 29, 2020
(86) PCT No.: PCT/KR2020/014934

§ 371 Date: Dec. 7, 2020

(30) Foreign Application Priority Data
Oct. 31, 2019 (KR) 10-2019-0138202

(51) Int. Cl. C12Q 1/6886; G01N 33/574

(52) U.S. Cl. CPC C12Q 1/6886; G01N 33/57423; C12Q 2600/106

(57) ABSTRACT

The present invention relates to a method of analyzing a corrected TMB and a method for predicting a response to immune checkpoint inhibitors in a cancer patient using the same. According to a method, a computer-readable recording medium and an analyzing apparatus, for providing information according to an aspect, since the corrected TMB of the present invention is markedly highly predictive of the response to cancer immunotherapy in the cancer patient, compared to the conventional TMB, a patient group predicted to show a therapeutic effect can be selected and an appropriate treatment can be administered, thereby alleviating pain and treatment costs from the cancer patient.

### TECHNICAL FIELD

The present invention relates to a new method for predicting an immunotherapy response.

### BACKGROUND ART

Cancer accounts for the highest death rate of Korean people, and there is a continuous demand for development of anti-cancer agents.

Development processes of anti-cancer agents will now be briefly reviewed. There have been chemical anti-cancer agents for attacking cells divided using characteristics of rapidly proliferating tumor cells, and target anti-cancer agents for attacking specific molecules of tumor cells or a signal transfer system, both of which, however, had several adverse side effects. Then, cancer immunotherapy capable of minimizing adverse side effects using in vivo innate immunity emerged.

Cancer immunotherapy refers to a cancer therapy approach or method of activating the immune system of a human body to cause the immune system to combat cancer cells. In the cancer immunotherapy, only cancer cells are attacked using the immune system, resulting in less side effects than existing anti-cancer treatment, and the memory and adaptiveness of the immune system are used, enabling long-term anti-cancer efficacy. The cancer immunotherapy that overcomes drawbacks of existing anti-cancer agents as stated above is receiving much attention as a new paradigm in cancer treatment, and Science Magazine chose cancer immunotherapy as the research of the year in 2013.

The cancer immunotherapy can be categorized into therapeutic antibody (Rituximab, etc.) for targeting a tumor antigen, an immune checkpoint inhibitor for reactivating an immune cell, and an immune cell therapy for directly administering an immune cell (Oiseth et al, 2017).

Recently, the immune checkpoint inhibitor has brought advancement of progressive cancer treatment. It has been found that antibodies targeting PD-1, PD-L1 and cytotoxic T-lymphocyte antigen-4 (CTLA-4) provide surprisingly improved viability in all kinds of cancers. However, only a small number of patients were able to benefit from the immune checkpoint inhibitor in spite of the potential, and more than half of patients experienced early worsening of disease. Therefore, demands for an effective biomarker for immune checkpoint inhibitor therapy are currently being emphasized.

Several existing studies have consistently put forward a possibility that a high tumor mutation burden (TMB) is associated with an immune checkpoint inhibitor (ICI) therapy in many kinds of cancers. Particularly, it has been revealed that a tumor specific neoantigen based on the immune system plays a role in activating an anti-tumor immune response. However, identifying patients who will benefit from ICI therapy remains unsolved in clinical usability of TMB. In fact, the distribution of TMB has been shown to substantially overlap between responsive and non-responsive tumors, indicating that TMB alone is not a sufficient biomarker for predicting a clinical success rate.

Existing studies barely elucidated the underlying mechanism for restricted effects of cancer immunotherapy. Recent genomic studies have figured out limitations of TMB and have explored other potential biomarkers to find influences of genomes associated with cancer immunotherapy. Actually, it was found that a corrected antigen presentation pathway became a basis of immunity evasion and eventually might have an impact on anti-tumor immunity, but clinical significance of inhibitory antigen presentation in the response to ICIs has not been clearly revealed yet.

Therefore, the present inventors analyzed sequencing data from 198 non-small cell lung cancer (NSCLS) tumors prior to immunotherapy, explored new predictive biomarkers in anti-tumor immune responses and investigated whether the explored biomarkers could be combined with existing biomarkers. In addition, in order to find out an association between the corrected antigen presentation pathway and ICIs, the present inventors introduced a new analysis method.

### DESCRIPTION OF EMBODIMENTS

### Technical Problem

An aspect provides a method for analyzing a corrected TMB, the method comprising the steps of: sequencing a biological sample obtained from a cancer patient; filtering data output from the sequencing; calculating a tumor mutation burden (TMB) using the filtered sequencing data; and correcting the calculated TMB using Equation 1:

$$Corrected TMB = TMB \times \frac{( NeoAg - NeoAgL + NeoAgC )}{NeoAg} . \qquad [ Equation 1 ]$$

Another aspect provides a method for providing information for predicting a response to cancer immunotherapy in a cancer patient with the calculated TMB.

Another aspect provides a computer-readable recording medium having a program recorded therein for executing the method on a computer.

Still another aspect provides an apparatus for analyzing a response to cancer immunotherapy in a cancer patient, the apparatus comprising: a data generation unit for generating a set of gene data by performing sequencing on a biological sample obtained from a cancer patient; a calculation unit for calculating a TMB by performing filtering the generated genetic data; and a correction unit for calculating the calculated TMB into a corrected TMB value using Equation 1.

### Solution to Problem

An aspect provides a method for analyzing a corrected TMB, the method comprising the steps of: sequencing a biological sample obtained from a cancer patient; filtering data output from the sequencing; calculating a tumor mutation burden (TMB) using the filtered sequencing data; and correcting the calculated TMB using Equation 1:

$$Corrected TMB = TMB \times \frac{( NeoAg - NeoAgL + NeoAgC )}{NeoAg} . \qquad [ Equation 1 ]$$

The TMB is the number of nonsynonymous alterations (single-nucleotide variations (SNVs) or indels), and NeoAg means a neoantigen burden calculated with Mupexi. In addition, NeoAgL is a value of neoantigens predicted to bind to lost HLA alleles as the output of Mupexi and LOH HLA, and is set to 0 in a case where there is no HLA LOH. NeoAgC is a value of neoantigens predicted to bind to both of lost HLA alleles and kept HLA alleles. When there was no HLA LOH, the value of NeoAgC was set to 0.

The corrected TMB value may also be referred to as HLA-corrected TMB.

The term “tumor mutation burden (TMB)” means a total number of mutations detected in cancer cell DNAs. In general, a cancer having many mutations may be highly responsive to a particular immunotherapy. The TMB or corrected TMB may be used as a biomarker for cancer immunotherapy.

The term “sequencing” may mean DNA or RNA sequencing. DNA sequencing may be determining a nucleotide sequence of DNA fragments. According to an embodiment, the DNA sequencing may mean a process of analyzing a sequence of DNA obtained from a biological sample of a cancer patient.

According to an embodiment, the sequencing method may be a chain termination method, pyrosequencing, next generation sequencing, deep sequencing or whole exome sequencing (WES). Preferably, the sequencing method is next generation sequencing, and the next generation sequencing is deep sequencing or whole exome sequencing (WES). According to a specific embodiment of the present invention, the sequencing method may be deep sequencing.

The term “deep sequencing” is also referred to as high-throughput sequencing and may mean that a genomic region of interest is sequenced multiple times. More specifically, deep sequencing may be sequencing performed 100 to 1000 times, and may means a next generation sequencing approach. In the case of using the deep sequencing method, rare gene defects of 1% or less of all gene defects, or clone types, may be detected.

In the case of the whole exome sequencing, which is a process of sequencing the whole gene sequences, this sequencing process may be an excessively time-consuming and costly process, and when the read depth is not so high, the sensitivity of whole exome sequencing may be undesirably lowered. In the case of using the deep sequencing, which is a kind of high depth targeted sequencing methods, the aforementioned drawbacks may be minimized.

FIG. 4 is a flow diagram of a method for predicting a response to a corrected TMB and immune checkpoint inhibitors using the same in a cancer patient, according to an aspect.

As shown in FIG. 4, the method may include a step of sequencing a biological sample obtained from a cancer patient. The cancer patient may be a lung cancer patient or a small cell lung cancer patient, and the biological sample may be a cancer cell. The step may include a step of analyzing gene sequences of the biological sample of the cancer patient.

As shown in FIG. 4, the method may include a step of filtering data output from the sequencing. In detail, the filtering may include a step of eliminating unnecessary information or parameters from the patient's gene sequencing result to calculate a TMB. The filtering may be basic filtering or additional filtering.

The term “filtering” means a step of correcting the sequencing data. More specifically, the filtering may be a step of increasing the accuracy by eliminating values not complying with the definition of the TMB. More specifically, the filtering may mean a correcting step, except for additionally calculated portions.

The filtering may be selected from the group consisting of non-coding region variant filtering, germline variant filtering, synonymous mutation filtering, Low VAF variant filtering, truncated mutation filtering, and known somatic alteration filtering.

The basic filtering may include eliminating or lowering non-coding region variants, germline variants, or synonymous mutations. The basic filtering may be a step of eliminating variants of a minor allele frequency (MAF), eliminating variants in non-coding regions, or eliminating synonymous mutations. The eliminating variants of a MAF is eliminating MAF mutation value greater than 1%. More specifically, the basic filtering may be a step of eliminating the variants having a minor allele frequency of 1% or more from Exome Aggregation Consortium (ExAC) database and 1000 Genome Project database on the assumption that the variants are germline variants, eliminating the variants discovered from non-coding regions, such as intron, etc., because a tumor mutation burden is calculated on the variants of coding regions, and then eliminating synonymous mutations among variants remaining after the above two eliminating steps because the synonymous mutations do not comply with the definition of tumor mutation burden.

The additional filtering may include a step of eliminating or lowering low VAF variants, truncated mutations or known somatic alteration. More specifically, first, the additional filtering may include a step of eliminating or lowering low VAF variants. The eliminating of the low VAF variants may be performed because the low VAF variants are highly probably variants of subclones of a tumor, and inclusion of the low VAF variants in calculating a TMB to predict a response to ICIs may create a confusion in the computation result. Next, the additional filtering may include a step of eliminating or lowering truncated mutations of tumor suppressor genes. Lastly, the additional filtering may include a step of eliminating or lowering tumor driven variants. When the method includes the additional filtering, the more accurate TMB computation may be performed through deep sequencing.

As shown in FIG. 4, the method may include a step of calculating a tumor mutation burden (TMB) using the filtered sequencing data. The TMB may be calculated with variables remaining after the filtering. The computation of the tumor mutation burden (TMB) may be performed using any of existing known calculating methods.

As shown in FIG. 4, the method may include a step of correcting the calculated TMB using Equation 1.

In an embodiment, the correcting may be a step of eliminating uncertainty due to neoantigen burden, etc. by correcting the existing TMB calculating methods, and consequently increasing the accuracy of TMB in a never-smoker lung cancer patient.

The term “neoantigen” means an antigen that has conventionally never been recognized in the immune system. The neoantigen may be derived from a tumor cell fragment, a tumor cell protein or virus protein derived from tumor cell mutation. More specifically, when mutation occurs to a protein coding gene of a cancer cell is mutated, the protein coding gene may be a potential neoantigen. In addition, the neoantigen may be measured by a next generation sequencing method. According to a specific embodiment, the neoantigen may be measured by deep sequencing.

The term “neoantigen burden” may mean a number of neoantigens and may be a kind of biomarkers predicting a response to ICIs. In addition, the neoantigen may be measured by sequencing.

According to an embodiment, the neoantigen may induce immune editing to LOA patients, and thus the TMB accuracy may be lowered. More specifically, the neoantigen may increase TMB scores by combining neoantigens with lost HLA genes.

The term “biological sample” means a sample obtained from a subject. The biological sample may include whole blood, plasma, serum, red blood cells, leukocytes (e.g., peripheral blood mononuclear cells), cannula, ascites, pleural efflux, nipple aspirate, lymphatic fluids (e.g., disseminated tumor cells of lymph nodes), bone marrow aspirates, saliva, urine, feces (i.e., excrement), sputum, bronchial washing liquids, tears, microneedle aspirates (e.g., randomly harvested by mammary microneedle aspiration), any other body fluids, tissue samples (e.g., tumor tissues) such as tumor biopsies (e.g., puncture biopsies) or lymph nodes (e.g., sentinel lymph node biopsy) such as surgical resection of tumors , and cell extracts thereof. In a specific embodiment, the biological sample may be whole blood or some constituents thereof, such as plasma, serum or cell pellets. In a specific embodiment, the sample may be obtained by isolating circulating cells of solid tumors from whole blood or cell fractions thereof using any technique known in the art. In a specific embodiment, the sample may be, for example, a formalin-fixed paraffin embedded (FFPE) tumor tissue sample from a solid tumor. In a specific embodiment, the sample may be a tumor lysate or extract prepared from frozen tissues obtained from a subject having a cancer.

The “obtaining” may mean obtaining a nucleic acid sample or a polypeptide sample from a biological sample. The obtaining of the nucleic acid sample may be performed by a general nucleic acid isolating method. For example, a target nucleic acid may be obtained by amplifying a nucleic acid by polymerase chain reaction (PCR), ligase chain reaction (LCR), transcription mediated amplification, or real time-nucleic acid sequence-based amplification (NASBA) and purifying the same. Alternatively, the target nucleic acid, which is an isolated nucleic acid, may be obtained from a lysate of the biological sample. The obtaining of the polypeptide sample may be performed by a general protein extracting or isolating method.

In a specific embodiment, the cancer patient may be a patient with lung cancer, melanoma, Hodgkin lymphoma, stomach cancer, urothelial cell cancer, head and neck cancer, liver cancer, colon cancer, prostate cancer, pancreatic cancer, liver cancer, testicular cancer, an ovarian cancer, endometrial cancer, cervical cancer, bladder cancer, brain cancer, breast cancer, or kidney cancer. For example, the cancer patient may be a lung cancer patient. Preferably, the cancer patient is a non-small cell lung cancer patient.

In a specific embodiment, the cancer patient may be a never-smoker or a former smoker. Preferably, the cancer patient is a never-smoker.

In a specific embodiment, the cancer of the cancer patient may be selected from the group consisting of lung cancer, melanoma, Hodgkin lymphoma, stomach cancer, urothelial cell cancer, head and neck cancer, liver cancer, colon cancer, prostate cancer, pancreatic cancer, liver cancer, testicular cancer, an ovarian cancer, endometrial cancer, cervical cancer, bladder cancer, brain cancer, breast cancer, and kidney cancer.

The lung cancer may be a non-small cell lung cancer or a small cell lung cancer.

In another aspect, provided is a method for providing information for predicting a response to cancer immunotherapy in a cancer patient, the method comprising the step of: sequencing a biological sample obtained from a cancer patient; filtering data output from the sequencing; calculating a tumor mutation burden (TMB) using the filtered sequencing data; correcting the calculated TMB using Equation 1; and providing the information for predicting the response to cancer immunotherapy in the cancer patient based on the calculated TMB.

In an embodiment, the predicting of the response may include predicting the response to be high when the corrected TMB value of the cancer patient is higher than that of a control group without a cancer, and to be low when the response is lower than that of the control group.

In an embodiment, the predicting of the response may include predicting the response to cancer immunotherapy in the cancer patient to be high when the corrected TMB value of the cancer patient is similar to or higher than that of a patient showing a good response to cancer immunotherapy, and to be low when the corrected TMB value is lower than that of the patient.

In an embodiment, the predicting of the response may include predicting the response to cancer immunotherapy in the cancer patient to be high when the corrected TMB value is top 20 to 30% or higher, and to be low when the corrected TMB value is bottom 20 to 30%, as compared with the whole patient data or general cancer patient data.

The cancer, the cancer patient, the obtaining, the biological sample, the sequencing, the filtering, the TMB, and the Equation 1 are the same as described above.

The term “cancer immunotherapy agent” may be an anti-cancer agent that kills cancer cells by activating immune cells of a human body and may mean a drug that shows a cancer treatment effect through patient's own immunopotentiation. In a specific embodiment, the cancer immunotherapy agent may be an immune checkpoint inhibitor.

The term “immune checkpoint inhibitor” may mean a cancer immunotherapy agent attacking a cancer cell by activating T cells by blocking an immune checkpoint protein involving T cell suppression, e.g., a protein such as PD-L1 expressed in a tumor cell. In a specific embodiment, the immune checkpoint inhibitor may be one selected from the group consisting of anti-PD-L1, anti-PD-1, and anti-CTLA-4, and may be, for example, anti-PD-1. In a specific embodiment, the immune checkpoint inhibitor may inhibit binding of PD-L1 and PD-1. Specifically, the immune checkpoint inhibitor may be an antibody capable of binding to PD-L1 or PD-1, such as a monoclonal antibody, and may be a human antibody, a humanized antibody, or a chimeric antibody.

The term “response to cancer immunotherapy” may mean whether or not a particular drug, e.g., an anti-cancer agent shows a therapeutic effect on an individual patient having a cancer. The term “predicting a response to cancer immunotherapy in a cancer patient” may mean that it is predicted before drug administration whether the drug administration is beneficial to treatment of a cancer, and may be predicting a response to the drug by measuring gene expression or using a biomarker.

The term “prediction” may mean previous determination of a particular result, e.g., a therapy response, by identifying a feature, such as an expression level of a particular gene or a biomarker.

The term “whole patient data” may mean TMB data of multiple groups of cancer patients. Specifically, the whole patient data may be data generated by a step of generating a set of gene data by performing sequencing on a biological sample obtained from the cancer patient.

In a specific embodiment, the predicting of the response may be identifying a distribution of the whole patient data and classifying the patients as a “high TMB group” or a “low TMB group” using a determined TMB cutoff. The TMB cutoff may define the patients as the “high TMB group” for top 30% and the “low TMB group” for bottom 30%. More specifically, the TMB cutoff for the high TMB group may be top 25%, top 20% or top 15%, and the TMB cutoff for the low TMB group may be bottom 25%, bottom 20% or bottom 15%.

In a specific embodiment, further increasing a reference point for cutting off the top TMB may contribute to increasing the accuracy in determining the TMB.

In an embodiment, the method may further include the steps of: measuring an expression level of programmed death-ligand 1 (PD-L1) from the biological sample obtained from the cancer patient; and predicting the response to cancer immunotherapy by combining the measured PD-L1 expression level with the corrected TMB value.

The predicting of the response may include evaluating that the higher the PD-L1 expression level, the better the response to ICIs, and even if the predicting of the response with PD-L1 alone may become incomplete, the predicting accuracy may be increased when the predicting of the response is determined by PD-L1 in combination with the corrected TMB value. More specifically, in the case of the low TMB group, the response may be more accurately predicted with PD-L1 expression level. For example, in the case of high TMB and high PD-L1 expression group, the response may be determined to be good or more, in the case of low TMB and low PD-L1 expression group, the response may be determined to be poor, and in the case of low TMB and high PD-L1 expression group, the extent of the response may be determined by identifying the PD-L1 value.

In a specific embodiment, the PD-L1 expression level may be measured by one or more techniques selected from the group consisting of western blot, enzyme linked immunosorbent assay (ELISA), radioimmunoassay (RIA), radioimmunodiffusion, Ouchterlony immunodiffusion, rocket immonoelectrophoresis, tissue immunity staining, immunoprecipitation assay, complement fixation assay, fluorescence activated cell sorter (FACS), and protein chip.

In a specific embodiment, a formulation for measuring an expression level of a protein may be an antibody specifically binding to the protein of the gene.

The term “antibody” is a term widely known in the related art and may mean a specific protein molecule indicated by an antigenic portion. An antibody according to an aspect is not particularly limited in its type, and examples thereof may include a polyclonal antibody, a monoclonal antibody or a portion thereof so long as it has an antigen binding property, and any immunoglobulin antibody, and may further include special antibodies, such as a humanized antibody, etc. The antibody according to an aspect may include not only a complete type of antibody having two full-length light chains and two full-length heavy chains but also a functional fragment of an antibody molecule. The functional fragment of an antibody molecule may mean a fragment having at least an antigen binding function, and may include, for example, Fab, F(ab′), F(ab′)2, or Fv.

The term “gene” may mean any nucleic acid sequence or a portion thereof which has a functional role in coding or transcribing a protein or in regulating expression of other genes. A gene may consist of any nucleic acid encoding a functional protein or only a portion of a nucleic acid encoding or expressing a protein. Nucleic acid sequences may include gene abnormalities in exons, introns, initiation or termination regions, promoter sequences, other regulatory sequences, or unique sequences adjacent to genes. A gene according to an aspect may be a tumor microenvironment gene. The term “tumor microenvironment (TME)” may mean a cellular environment in which cancer cells are surrounded by blood vessels, immune cells, fibroblasts, bone marrow derived inflammatory cells, lymphocytes, signal transmitting molecules, extracellular matrix, etc. The term “tumor microenvironment gene” is a gene involving a tumor microenvironment and may be used as a biomarker for predicting a response to cancer immunotherapy.

Another aspect provides a computer-readable recording medium having a program recorded therein for executing the method on a computer.

Another aspect provides a method for operating a computing apparatus processed by at least one processor, the method comprising a program for executing the method on a computer.

Another aspect provides a computing apparatus for analyzing a response to cancer immunotherapy in a cancer patient, the computing apparatus comprising a memory and at least one processor for executing instructions of a program loaded on the memory, the program comprising instructions described to execute the steps of: generating a set of gene data by performing sequencing on a sample obtained from the cancer patient; measuring a TMB by filtering the generated gene data; and correcting the measured TMB into a corrected TMB value using Equation 1.

FIG. 1 is a schematic diagram of a cancer patient response analyzing apparatus or a computing hardware and process according to an aspect.

Referring to FIG. 1, the analyzing apparatus 10 may derive a corrected TMB value using a biological sample obtained from the cancer patient and, on the basis of the corrected TMB value, may predict a response to cancer immunotherapy in the cancer patient.

FIG. 3 is a block diagram showing hardware components of a cancer patient response analyzing apparatus or a computing apparatus according to an aspect.

The gene data set received from the data generation unit 110 of the analyzing apparatus 10 may be generated by performing sequencing on the biological sample obtained from the cancer patient.

Definitions and explanations of corrected TMB, Equation 1, cancer immunotherapy, cancer, cancer patient, biological sample, sequencing, and gene, are the same as described above.

Still another aspect provides an apparatus for analyzing a response to cancer immunotherapy in a cancer patient, the apparatus comprising: a data generation unit for generating a set of gene data by performing sequencing on a biological sample obtained from a cancer patient; a calculation unit for calculating a TMB by performing filtering the generated genetic data; and a correction unit for calculating the measured TMB into a corrected TMB value using Equation 1.

In an embodiment, the apparatus may further include an analysis unit for predicting the response to cancer immunotherapy in the cancer patient to be high when the corrected TMB value of the cancer patient is higher than that of a control group without a cancer, and to be low when the corrected TMB value is lower than that of the control group.

FIG. 1 is a schematic diagram of a cancer patient response analyzing apparatus according to an aspect.

Referring to FIG. 1, the cancer patient response analyzing apparatus 110 may derive a corrected TMB value using a biological sample obtained from the cancer patient and, on the basis of the corrected TMB value, may predict a response to cancer immunotherapy in the cancer patient.

FIG. 3 is a block diagram showing hardware components of a cancer patient response analyzing apparatus according to an aspect.

The gene data set received from the data generation unit 110 of the cancer patient response analyzing apparatus 10 may be generated by performing sequencing on the biological sample obtained from the cancer patient. The biological sample and the sequencing are the same as described above.

In the cancer patient response analyzing apparatus 10, the calculation unit 120 may receive the gene data set, perform filtering on the received gene data and measure a TMB. The filtering and the TMB measuring are the same as described above.

In the cancer patient response analyzing apparatus 10, the correction unit 130 may calculate the measured TMB into a corrected TMB value using Equation 1. The Equation 1 and the corrected TMB value are the same as described above.

In an embodiment, the apparatus may further include an analysis unit for predicting the response to cancer immunotherapy in the cancer patient to be high when the corrected TMB value is similar to or higher than that of a patient showing a good response to cancer immunotherapy, and to be low when the corrected TMB value is lower than the patient.

The analysis unit may identify a distribution of the whole patient data and may classify the patients as a “high corrected TMB group” or a “low corrected TMB group” using a determined TMB cutoff. In addition, the TMB cutoff may define the patients as the “high TMB group” for top 30%, and the “low TMB group” for bottom 30%. More specifically, the TMB cutoff for the high TMB group may be top 25%, top 20% or top 15%, and the TMB cutoff for the low TMB group may be bottom 25%, bottom 20% or bottom 15%.

In an embodiment, when the corrected TMB value is top 30 to 0%, as compared with the whole patient data, the apparatus may further include an analysis unit for defining the cancer patient to be classified as a high TMB group and predicting the response to cancer immunotherapy in the cancer patient to be high. For example, the corrected TMB value may be top 30 to 0%, 25 to 0%, 20 to 0%, 15 to 0% or 10 to 0%. Preferably, the corrected TMB value is 20 to 0%.

In an embodiment, when the corrected TMB value is bottom 30 to 0%, the apparatus may further include an analysis unit for defining the cancer patient to be classified as a low TMB group and predicting the response to cancer immunotherapy in the cancer patient to be low. For example, the corrected TMB value may be bottom 30 to 0%, 25 to 0%, 20 to 0%, 15 to 0% or 10 to 0%. Preferably, the corrected TMB value is 20 to 0%.

In a specific embodiment, further increasing a reference point for cutting off the top TMB may contribute to increasing the accuracy in determining the TMB.

In an embodiment, the apparatus may further include: a measuring unit for measuring a PD-L1 expression level from the biological sample obtained from the cancer patient; and a determination unit for determining a response to cancer immunotherapy by combining the measured PD-L1 expression level with the corrected TMB value.

The determination unit may evaluate that the higher PD-L1 expression level, the higher the response to ICIs, and even if the predicting of the response with PD-L1 alone may become incomplete, the predicting accuracy may be increased when the prediction of the response is determined by PD-L1 in combination with the corrected TMB value. More specifically, in the case of the low TMB group, the response may be more accurately predicted with PD-L1 expression level. For example, the response may be determined to be good in cases of high TMB and high PD-L1 expression, and to be poor in cases of low TMB and low PD-L1 expression, while an extent of the response may be determined by identifying the PD-L1 value in cases of low TMB and high PD-L1 expression.

In a specific embodiment, the PD-L1 expression level may be measured by one or more techniques selected from the group consisting of western blot, enzyme linked immunosorbent assay (ELISA), radioimmunoassay (RIA), radioimmunodiffusion, Ouchterlony immunodiffusion, rocket immonoelectrophoresis, tissue immunity staining, immunoprecipitation assay, complement fixation assay, fluorescence activated cell sorter (FACS), and protein chip.

In a specific embodiment, a formulation for measuring an expression level of the protein may be an antibody specifically binding to the protein of the gene.

In a specific embodiment, the cancer patient may be a lung cancer patient, a melanoma patient, a Hodgkin lymphoma patient, a stomach cancer patient, a urothelial cell cancer patient, a head and neck cancer patient, a liver cancer patient, a colon cancer patient, a prostate cancer patient, a pancreatic cancer patient, a liver cancer patient, a testicular cancer patient, an ovarian cancer patient, an endometrial cancer patient, a cervical cancer patient, a bladder cancer patient, a brain cancer patient, a breast cancer patient, or a kidney cancer patient. For example, the cancer patient may be a lung cancer patient. Preferably, the cancer patient is a non-small cell lung cancer patient.

In a specific embodiment, the cancer patient may be a never-smoker or a former smoker. Preferably, the cancer patient is a never-smoker, but not limited thereto.

In a specific embodiment, the cancer of the cancer patient may be selected from the group consisting of lung cancer, melanoma, Hodgkin lymphoma, stomach cancer, urothelial cell cancer, head and neck cancer, liver cancer, colon cancer, prostate cancer, pancreatic cancer, liver cancer, testicular cancer, ovarian cancer, endometrial cancer, cervical cancer, bladder cancer, brain cancer, breast cancer, and kidney cancer.

The lung cancer may be a non-small cell lung cancer or a small cell lung cancer.

The obtaining, the biological sample, the sequencing, the filtering, the TMB, the Equation 1, the cancer immunotherapy, the response and the expression may be the same as described above.

The terms used in various examples of the present invention have been selected as general terms currently used as widely as possible in the art in consideration of functions in the present embodiments, but these terms may vary according to the intention or precedent cases of a person skilled in the art, the advent of new technologies. Also, in certain cases, there may be an arbitrarily selected term, in which case the meaning thereof will be described in detail in the description of the corresponding embodiment. Therefore, the term used in various examples of the present invention should be defined based on the meaning of the term rather than the name of the term, and the contents of the present invention throughout the present invention.

In descriptions of the present embodiments, when regions or components are referred to as being connected to each other, they can be directly connected to each other or can be indirectly electrically connected to each other due to other intervening regions or components being present therebetween. In addition, when a component is referred to as comprising another component, this specifies the inclusion of one or more other components, but does not preclude the possibility of excluding the stated components In addition, the terms “˜unit”, “˜module” used in various embodiments may mean a unit that processes at least one function or operation, which can be implemented by hardware or software or a combination of hardware and software.

As used herein, the terms “consisting of” or “comprising” should not be construed as necessarily including the various elements or steps described in the specification, or may be further comprised of additional components or steps.

The description of the following embodiments should not be construed as limiting the scope of rights, and it should be construed as belonging to the scope of the embodiments as can be easily inferred by those skilled in the art. Hereinafter, only exemplary embodiments will be described in detail with reference to the accompanying drawings. Hereinafter, only exemplary embodiments will be described in detail with reference to the accompanying drawings,

### Advantageous Effects of Disclosure

According to the method for predicting for predicting a response to a corrected TMB and immune checkpoint inhibitors using the same in a cancer patient, according to an aspect, since the corrected TMB of the present invention is markedly highly predictive of the response to cancer immunotherapy in the cancer patient, a patient group predicted to show a therapeutic effect can be selected and an appropriate treatment can be administered, thereby alleviating pain and treatment costs from the cancer patient.

### BRIEF DESCRIPTION OF DRAWINGS

FIG. 1 is a schematic diagram of a cancer patient response analyzing apparatus according to an aspect.

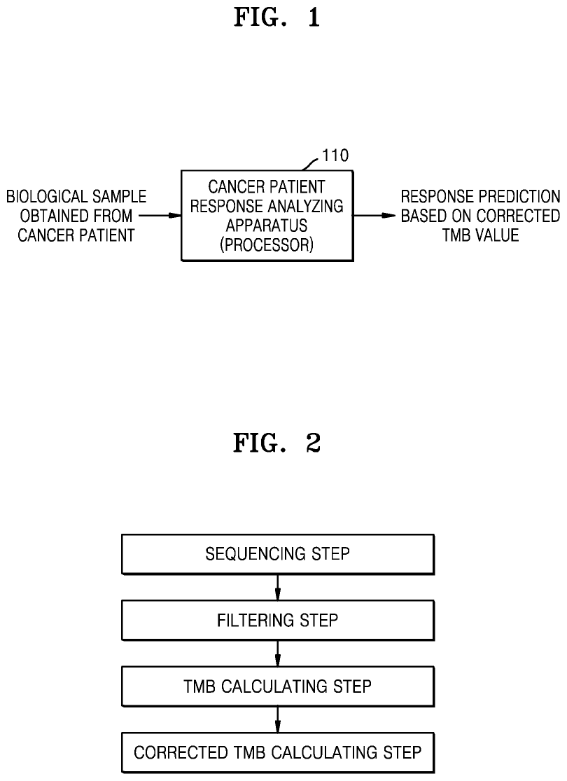

FIG. 2 is a flow diagram of a method for predicting a response to a corrected TMB according to an aspect and immune checkpoint inhibitors using the same in a cancer patient.

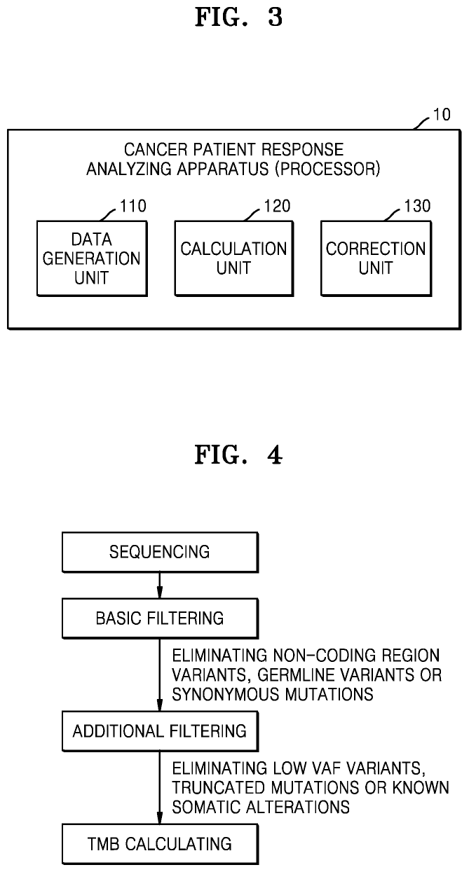

FIG. 3 is a block diagram showing hardware components of the cancer patient response analyzing apparatus according to an aspect.

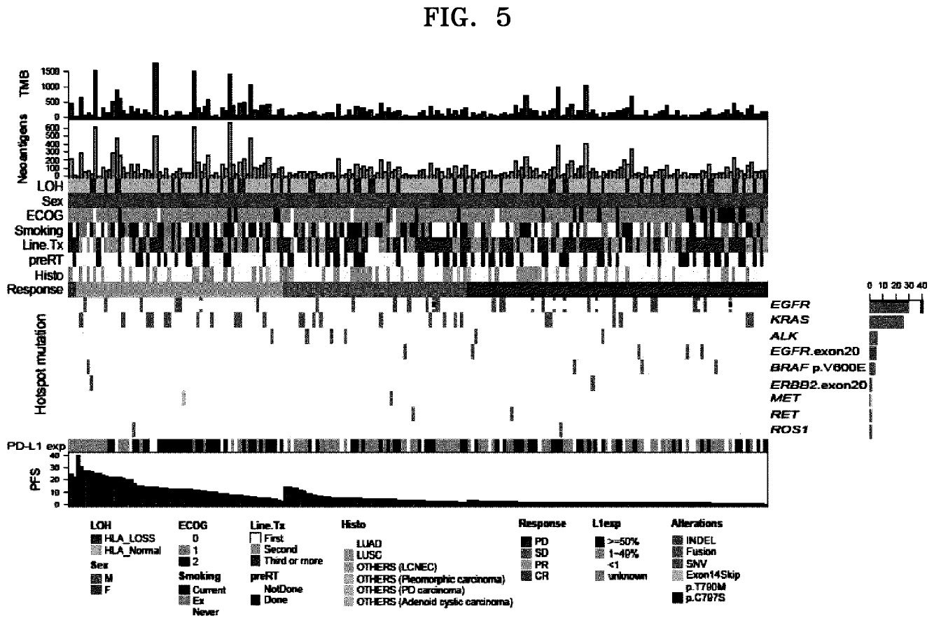

FIG. 4 is a flow diagram showing a filtering process in a method for predicting a response to cancer immunotherapy in a cancer patient, according to an aspect.

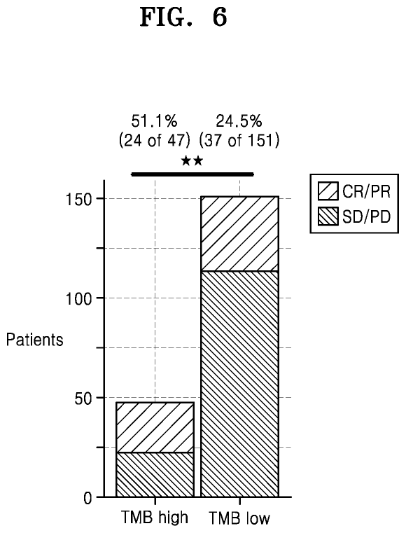

FIGS. 5 to 9 are diagrams showing clinical and genetic features associated with responses to TMB and ICIs in NSCLC patients as a study cohort.

FIG. 5 is a heat map of oncogenic drivers identified from NSCLC patient tumors sampled before immunotherapy, and each column represents individual patients grouped according to clinical response.

FIG. 6 is a graph showing ORR to ICI in patients with high TMB versus low TMB (using Fisher's exact test).

FIG. 7 shows graphs illustrating genetic and clinical features associated with TMB (from the left plot, P=0.0105 for oncogenic driver mutation versus wide type, P<0.0001 for current smokers versus never-smokers, P<0.0001 for former smokers versus never smokers, P=0.2332 for current smokers versus former smokers, and P<0.0001 for HR alteration versus wild type, and Mann-Whitney U test was used).

FIG. 8 is a graph representing PFS on ICIs stratified by TMB high and TMB low.

FIG. 9 is a graph for Kaplan-Meier survival analysis showing that HR deficiency is associated with increased PFS in patients treated with ICIs, in which P value indicates two-sided log-rank test.

FIGS. 10 to 13 are graphs representing differences in the biomarkers between smokers and never-smokers.

FIG. 10 is a graph for comparison of TMB and PD-L1 expression between response and non-response groups after patient groups are classified according to whether the patients are smokers or never-smokers.

FIG. 11 is a graph showing the comparison result of PD-L1 expressing patients.

FIG. 12 shows nonsynonymous mutations associated with a response to ICIs in the never-smoker group, in which only genes having a Q value of 0.01 or less were measured.

FIGS. 13A and 13B are graphs showing PFS or EGFR alteration due to TP53 in the never-smoker group treated with ICI.

FIG. 14A shows frequencies of HLA LOH events in patient groups.

FIG. 14B shows an association between HLA intact and HLA LOH in patient groups.

FIG. 14C shows a comparison of the HLA intact and the HLA LOH, which is not related to the elevated ORR, but is related to the corrected TMB.

FIG. 15 shows neoantigens for each patient, predicted to bind to the lost HLA LOH or the kept HLA LOH.

FIG. 16A shows an association between corrected TMB and HLA LOH in patient groups.

FIG. 16B shows PFS in patient groups classified by HLA LOH status.

FIGS. 17A and 17B show proportions of nonsynonymous mutations binding only to the lost HLA alleles and only to the kept alleles.

FIG. 18A shows a distribution of patient groups changed in subgroup by corrected TMB from a high TMB group to a low TMB group.

FIG. 18B shows relapse or death rates of patient groups changed in subgroup by corrected TMB from a high TMB group to a low TMB group.

FIGS. 19A and 19B show relapse or death rates of patient groups reclassified as a high TMB group by corrected TMB.

FIG. 20A shows a distribution of TMB values in the whole patient cohort by corrected TMB.

FIG. 20B shows a distribution of TMB values in subgroup changed patient cohorts by corrected TMB.

FIGS. 21A and 21B show time-dependent relapse or death risks of subgroup changed patient cohorts by corrected TMB.

FIG. 22A shows distributions of PD-L1 expression of CR/PR (n=45) versus SD/PD (n=99) patients and PD-L1 expression of high TMB (n=35) versus low TMB (n) patients.

FIG. 22B shows responses to ICIs in TMB high group.

FIG. 22C shows responses to ICIs in TMB low group.

FIG. 22D shows percentages of responses in patient groups classified by TMB and PD-L1.

FIG. 23 shows Forest plots representing models of multivariate cox proportional hazards.

### MODE OF DISCLOSURE

Hereinafter, the present invention will be described in more detail through examples. However, these examples are provided only for illustration of the present invention and the scope of the present invention is not limited to these examples.

### Example 1

### Experimental Materials and Preparation of Experiments

1.1 Methods for Patient Preparation and Response Evaluation

Studies were conducted on 198 non-small cell lung cancer patients. In detail, the patients consisted of a patient group (77%) treated with anti-PD-1 inhibitor single therapy and a patient group (23%) treated with anti-PD-1 single therapy. Before starting ICI treatment, biopsy tissue samples were sampled from the patients. In addition, only when valid data for efficacy analysis were obtained from the patients, the patients were eligible for enrollment in the experiments.

Table 1 shows data of 198 non-small cell lung cancer patients participated in experiments.

TABLE 1

| Patient | All Patients | | CR/PR | | SD/PD | | p Value |
|---|---|---|---|---|---|---|---|
| N = 198 | No. | (%) | 61 | (31%) | 137 | (69%) | |
| | | | No. | (%) | No. | (%) | |
| Median Age (range) | 62.1 | (33-84) | 65.1 | (44-83) | 61.4 | (33-84) | 0.0236 |
| Gender | | | | | | | 0.3528 |
| Male | 140 | (71) | 48 | (75) | 94 | (69) | |
| Female | 58 | (29) | 15 | (25) | 43 | (31) | |
| Median TMB (range) | 143 | (1-1765) | 194 | (3-1765) | 131 | (1-1035) | 0.0069 |
| Smoking status | | | | | | | 0.1492 |
| Current/former | 130 | (66) | 45 | (74) | 85 | (62) | |
| Never | 68 | (34) | 16 | (26) | 52 | (38) | |
| Performance status | | | | | | | 0.2526 |
| ECOG 0 & 1 | 172 | (87) | 56 | (92) | 116 | (85) | |
| ECOG 2 | 26 | (13) | 5 | (8) | 21 | (15) | |
| Histology | | | | | | | 0.9664 |
| LUAD | 129 | (65) | 40 | (66) | 89 | (65) | |
| LUSC | 58 | (29) | 18 | (29) | 40 | (29) | |
| Others | 11 | (6) | 3 | (5) | 8 | (6) | |
| Immunotherapy | | | | | | | 0.5976 |
| Nivolumab | 74 | (37) | 20 | (33) | 54 | (40) | |
| Pembrolizumab | 78 | (40) | 27 | (44) | 51 | (37) | |
| Anti-PDL-1 agent | 46 | (23) | 14 | (23) | 32 | (23) | |
| Line of therapy | | | | | | | 0.3931 |
| First | 14 | (7) | 5 | (8) | 9 | (6) | |
| Second | 66 | (33) | 24 | (39) | 42 | (31) | |
| Third or more | 118 | (60) | 32 | (53) | 86 | (63) | |
| PD-1 expression | | | | | | | 0.0209 |
| <1% | 34 | (17) | 8 | (13) | 26 | (19) | |
| 1-49% | 35 | (18) | 6 | (10) | 29 | (21) | |
| ≥50% | 75 | (38) | 31 | (51) | 44 | (32) | |
| Unknown | 54 | (27) | 16 | (26) | 38 | (28) | |

| | | | |
|---|---|---|---|
| Actionable mutations | | | |
| EGFR mut^{a} | 36 | 7 | 29 |
| KRAS mut^{b} | 26 | 11 | 15 |
| ALK fusion | 6 | 1 | 5 |
| BRAF mut (p. V600E) | 4 | 1 | 3 |
| ROS1 fusion | 2 | 1 | 1 |
| RET fusion | 2 | 0 | 2 |

The abbreviations used in Table 1 are as follows: CR for complete response; PR for partial response; SD for stable disease; PD for progressive disease; ECOG for The Eastern Cooperative Oncology Group; LUAD for lung adenocarcinoma; LUSC for lung squamous cell carcinoma; and HLA LOH for a loss of heterozygosity at the class I human leukocyte antigen. In addition, aEGFR mutation stands for De119, L858R, Del18, or Ins20, and bKRAS mutation stands for G12A, G12C, G12D, or G12V.

As indicated in Table 1, target response proportion for single administration of ICI (complete response (CR)/partial response (PR)) was 31%. The median TMB was 143 mutations (range: 1 to 1765 mutations), and the TMB distribution was similar to that of non-small cell lung cancer patients treated with ICIs in a previous study. 184 patients among 198 patients in total, that is, 93%, were administered subsequent systemic anti-cancer therapies. ECOG (Eastern Cooperative Oncology Group) scores of response and non-response groups were similar. However, it was confirmed that patients with an ECOG score of 2 were 26, i.e., 13%, showed a significantly short progression free survival (PFS) (hazard ratio (HR)=2.43, 95% confidence interval (CI)). The other different clinical features were evenly distributed between the objective response (OR) group and the non-response group.

The above-mentioned research preparation was approved by Samsung Medical Center Institutional Review Board/Personal Information Protection Commission (Approval numbers: 2018-03-130 and 2013-10-112) or Seoul National University Hospital Biomedical Research Institute (Approval number: 1805-109). All participant patients offered a written consent before enrollment, and objective responses were evaluated using Response Evaluation Criteria in Solid Tumors, version 1.1 (RECIST v1.1). More specifically, CR or PR patients were assessed as responders, and SD or PD patients were assessed as non-responders. Statistical features of patient groups were obtained from electronic medical records. The PD-L1 expression in a sample was assessed by US FDA-approved Dako PD-L1 IHC 22C3 pharmDx kit (Agilent Technologies).

1.2 Tissue Genomic Analysis

All tumor samples (FFPE and fresh frozen tissues) were sampled before ICI treatment. DNA extraction, library preparation and sequencing were processed in the same manner as described above, total genomic DNA was extracted by using DNeasy blood and tissue kit (Qiagen, 69504), QIAamp DNA FFPE tissue kit (Qiagen, 56404) or AllPrep DNA/RNA Mini kit (Qiagen, 80204). The presence of tumor tissues predicted to exist in the sequenced samples and the percentages of tumors predicted to survive were reviewed by a thoracic pathologist (Y.L.C). The mean sequencing coverages across all tumor samples and blood samples were 100×and 50×, respectively, and samples in the poor sequencing coverage (average target range of tumors<25×, average target range of normal tissues<15×) were excluded. BAM files to be analyzed were generated using a pipeline to be described later. To identify tumor/normal tissue pairs, NGSCheckMate was used in a quality control (QC) step. Somatic mutations were discriminated by comparing tumor and matched peripheral blood mononuclear cell blood samples using MuTect 2 and Pindel algorithms. In addition, copy numbers and B-allele frequency profiles were constructed using Control-FREEC.

1.3 Evaluation of HR Genes and HR Deficiency

HR pathway related genes discovered in previous studies (data not shown) were used, and tumors having truncated mutations including deletions, stop-gain mutations, and frameshifts or splice site alterations in the HR pathway related genes, were considered to be HR deficient. To identify mutations in BRCA1 and BRCA2, all variants were manually reviewed according to the standards and guidelines provided by the American Medical College of Medical Genetics and Genomics (ACMG).

1.4 Definitions of Significantly Mutated Genes and Immune-Related Genes

Significantly mutated genes were identified in 198 patients with NSCLC using MutSigCV. In this experiment, a significant mutation was defined as a mutated gene with a Q-value of less than 0.01 in the MutSigCV algorithm. 12 genes reported as predictive factors in a previous study, including STK11, JAK2, JAK1, B2M, TAP1, TAP2, TAPBP, CANX, HSPAS, PDIA3, CALR, and POLE, were used as the immunotherapy related genes, and survival analysis was limited to one or more genes.

1.5 HLA Analysis and Silico Neoantigen Prediction Pipeline

Digit class HLA-I was obtained from germline WES reads using Optitype. Specifically, MuTect2 was used to generate a list of mutant peptides, and neoantigen prediction was performed with MuPeXI. The MuPeXI was used to run NetMHCpan v4.0, and all novel 9-mer mutant peptides were computed from detected somatic mutations consisting of point mutations, insertions and deletions, using MuPeXI. Predicted percentile rank affinity scores were determined by NetMHCpan-4.0 for both mutant and normal peptides. MuPeXI rank mutant peptides were based on priority scores. The priority scores were calculated in the following manner. First, percentile rank based affinities of mutations and normal peptides were predicted, and reference proteome matching penalty and mutation allele frequency were calculated. In addition, since considerable portions of feasible variants may have a low allele frequency (AF) and variable AFs within a section of identical clinical samples, it was assumed in this study that mutant AF was set to an equal constant.

1.5 HLA Analysis and Silico Neoantigen Prediction Pipeline

A halplotype-specific copy number of the HLA locus was calculated using LOH HLA, and purity and polyploidy estimates of CHAT were used as input values.

1.6 Statistical Analysis

In the present invention, progression free survival (PFS) and overall survival (OS) were estimated using the Kaplan-Meier method, and differences between groups in PFS and OS were assessed using the log-rank test. In addition, categorical variables between two groups were compared using the Fisher's exact test, or chi-square test for three groups. Differences in means or medians for a continuous variable between two groups were assessed by the non-parametric Mann-Whitney U test or unpaired t test, and Benjamini-Hochberg P value was used in explaining multiple comparisons. Hazard ratio (HR) and 95% confidence interval (CI) were computed using the Cox proportional hazards model. The multivariate survival analysis was performed using the Cox proportional hazards model to assess the impact of TMB, PD-L1 expression and HR gene alteration on PFS while adjusting other covariates. A receiver operating characteristic (ROC) curve plotting sensitivity and 1-specificity of continuous variables was used. In addition, P values less than 0.05 were considered to indicate a statistically statistical significance for all comparisons, and the P values were all two-sided. All statistical analyses were performed using software R 3.3.3.

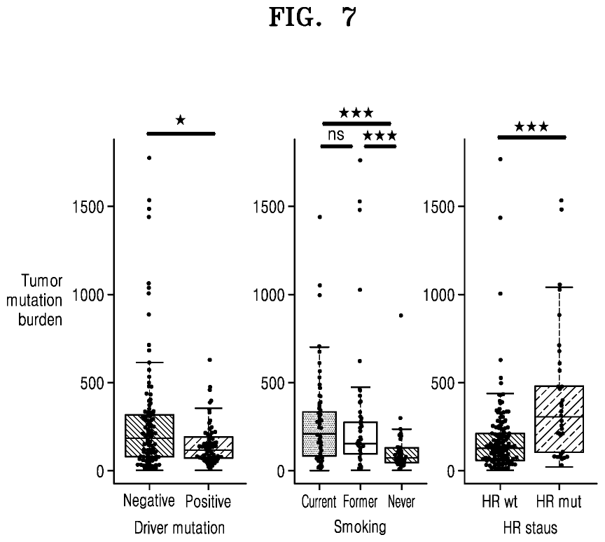

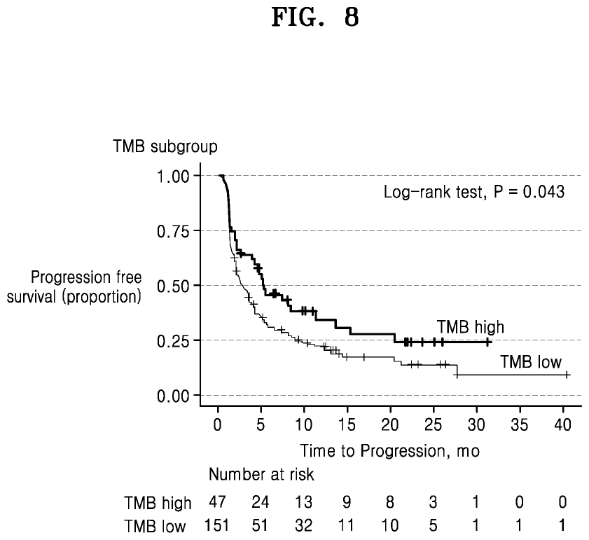

FIG. 5 is a heat map of oncogenic drivers identified from NSCLC patient tumors sampled before immunotherapy, and each column represents individual patients grouped according to clinical response.

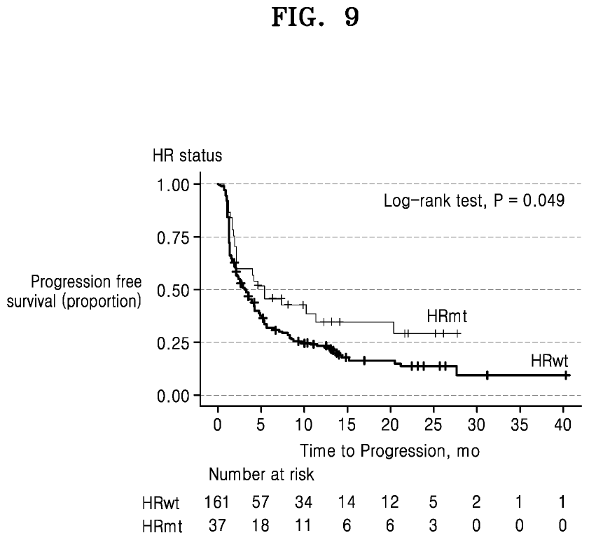

FIG. 6 is a graph showing ORR to ICI in patients with high TMB versus low TMB (using Fisher's exact test).

As shown in FIGS. 5 and 6, the results showed that clinical and genetic characteristics of the group of 198 patients as experimental subjects, associated with the TMB and the response to ICIs, were analyzed.

### Example 2

### Calculating Method of Corrected TMB

General used Silico neoantigen prediction is based on peptide-HLA binding affinity, and the prediction accuracy of the method may be somewhat improved by accumulating eluted ligand data. Neoantigen burden measurement has hitherto been performed through computer-based prediction, which was, however, problematic in accuracy, and was inaccurate in predicting a response to ICIs. Therefore, the present inventors determined that TMB was more adequate for predicting a response to ICIs. For the foregoing reasons, in the present invention, HLA-corrected TMB of the following equation was designed, instead of using the neoantigen burden:

$$Corrected TMB = TMB \times \frac{( NeoAg - NeoAgL + NeoAgC )}{NeoAg} \qquad [ Equation 1 ]$$

wherein TMB is the number of nonsynonymous alterations (single-nucleotide variations (SNVs) or indels),

NeoAg is a neoantigen burden calculated with Mupexi, and

NeoAgL is a value of neoantigens predicted to bind to lost HLA alleles as the output of Mupexi and LOH HLA. In a case where there is no HLA LOH HLA LOH, the NeoAgL value was set to 0.

NeoAgC is a value of neoantigens predicted to bind to both of lost HLA alleles and kept HLA alleles. In a case where there is no HLA LOH HLA LOH, the NeoAgC value was set to 0.

### Example 3

### Identification of Association Between Cause and Response to ICIs in Non-Small Cell Lung Cancer (NSCLC) Patient

3.1 Determination of Need for Correcting TMB

The result of measuring TMB values in the patient groups of Example 1 showed that the TMB values in the OR group were larger than those in the non-response group. The present inventors defined “high TMB” in many studies using a variable cutoff of TMB. First, the cohort of non-small cell lung cancer patients treated with ICIs was classified by the percentile value to define 25 TMB bottom groups, and this approach was also used in determining a reference for defining “high TMB”.

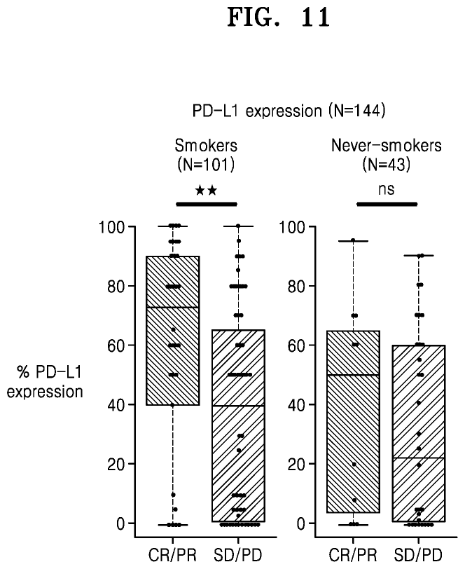

FIG. 8 is a graph representing PFS on ICIs stratified by TMB high and TMB low.

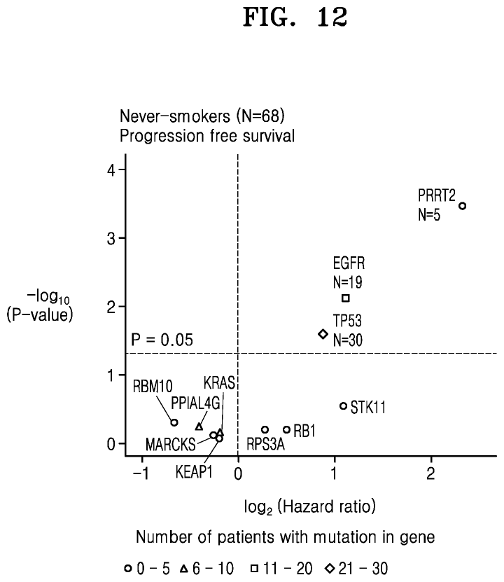

FIG. 9 is a graph for Kaplan-Meier survival analysis showing that HR deficiency is associated with increased PFS in patients treated with ICIs, in which P value indicates two-sided log-rank test.

Referring to FIGS. 8 and 9, there was an association between high TMB (272 mutations was designed as a high TMB group defined as top 25% in independent study cohorts) and improved progression free survival (PFS) (HR=0.67, 95% CI, 0.45-0.99, log-rank test, P=0.043). However, the area under the curve (AUC) was 0.62 (95%), which means that TMB, when used alone, was a very inferior biomarker.

3.3 Identification of Associations Between Homology-Dependent Recombination Gene Alterations and Increased TMB and Therapeutic Effect of ICI Treatment

Next, the present inventors investigated whether TMB was associated with other genetic and clinical features.

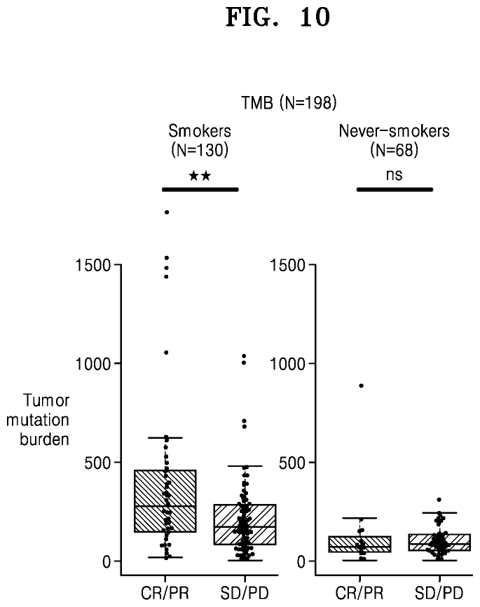

FIG. 7 shows graphs illustrating genetic and clinical features associated with TMB (from the left plot, P=0.0105 for oncogenic driver mutation versus wide type, P<0.0001 for current smokers versus never-smokers, P<0.0001 for former smokers versus never smokers, P=0.2332 for current smokers versus former smokers, and P<0.0001 for HR alteration versus wild type, and Mann-Whitney U test was used).

Referring to 7, the currently or formerly smoking status and homologous-recombination (HR) deficiency were associated with TMB values, while mutations as representative cancer genes were associated with a low TMB value.

In the whole patent cohort, HR deficiency was observed from 37 patients (18.7%), and the presence of HR deficiency was associated with longer PFS in the Kaplan-Meier survival analysis (HR=0.65, 95% CI, 0.42-1.00, log-rank test, P=0.049). However, an ample amount of data enough to confirm an association between OR versus ICIs (odds ratio=1.70, P=0.17) and HR deficiency was not available. In order to clarify the association between the response to ICIs and HR deficiency, the present inventors investigated whether the association was statistically significant in multivariate models. PD-L1 expression was observed in 144 patients. Altered HR function (27 out of 144, 18.8%) remained as an independent prediction variable of the response to ICIs (data not shown) after adjusting for age, sex, ECOG, histology, smoking and PD-L1 expression in these patients (HR 0.58, 95% CI, 0.34-0.99, P=0.046, data not shown).

3.4 Association Between Somatic Mutations and Response to ICIs in Never-Smoker Non-Small Cell Lung Cancer (NSCLC) Patients

In order to closely examine the genetic aspect of the response to ICIs, the present inventors investigated whether or not somatic mutations and copy number alterations (CNAs) contribute to the response to ICIs (data not shown). First, mutation genes were analyzed using 12 genes used as prediction biomarkers in a previous study, and MutSigCV. STK11 mutations were detected in nine patients, but no statistical significance was observed. In addition, four truncated mutations were identified in nine patients, and three among the nine patients showed no responses. In addition, the JAK1, JAK2, or B2M mutations were too rare to investigate their association with the response to ICIs. That is, only three patients out of all patients had JAK1, JAK2, or B2M mutation. CNa altherations were also investigated, but no significant association was found.

Since the study cohorts in this experiment included 34.4% of never-smokers, which is higher than in previous studies, the present inventors investigated whether never-smokers involved in the response to ICIs through other molecular mechanisms.

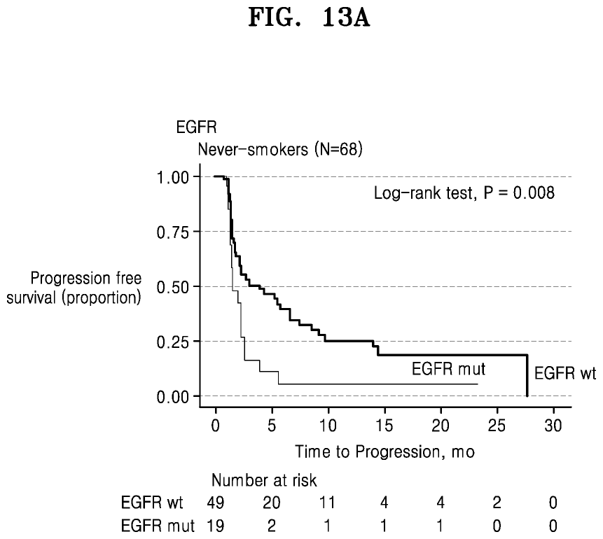

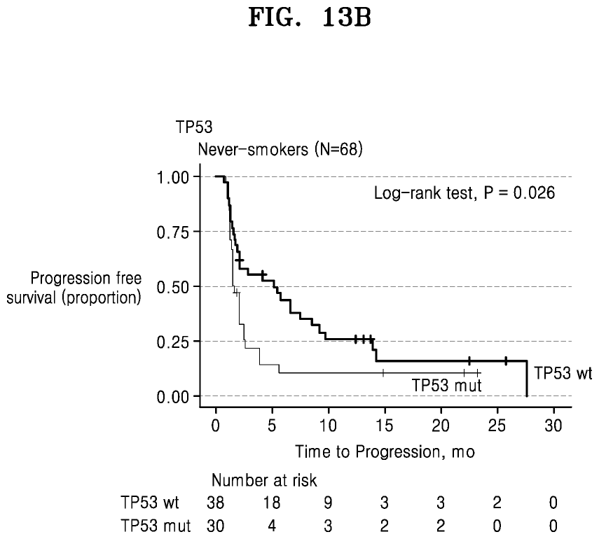

FIG. 10 is a graph for comparison of TMB and PD-L1 expression between response and non-response groups after patient groups are classified according to whether the patients are smokers or never-smokers.

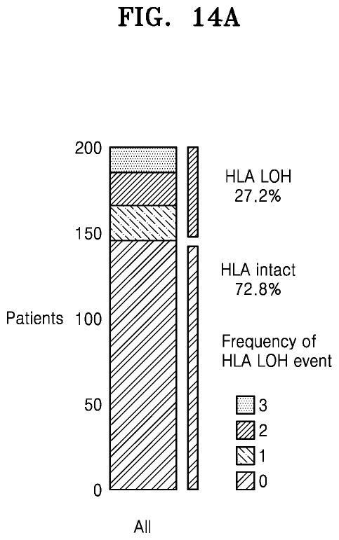

FIG. 11 is a graph showing the comparison result of PD-L1 expressing patients.

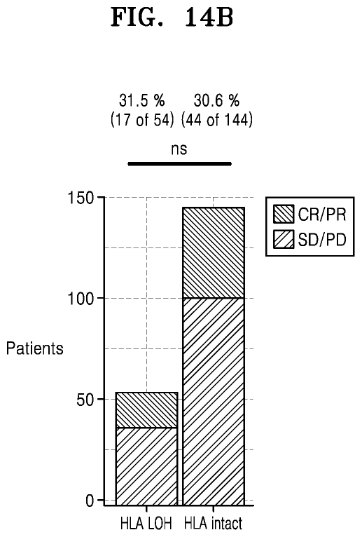

FIG. 12 shows nonsynonymous mutations associated with a response to ICIs in the never-smoker group, in which only genes having a Q value of 0.01 or less were measured.

Referring to FIGS. 10 and 12, in a case of never-smokers, it was confirmed that TMB and PD-L1 expression were not statistically significant in responders to ICI treatment, compared to non-responders to ICI treatment. This means that currently widely used biomarkers may not be effective to never-smokers.

Therefore, the present inventors additionally analyzed as to whether a mutation of a particular gene could act as a potential biomarker for never-smokers.

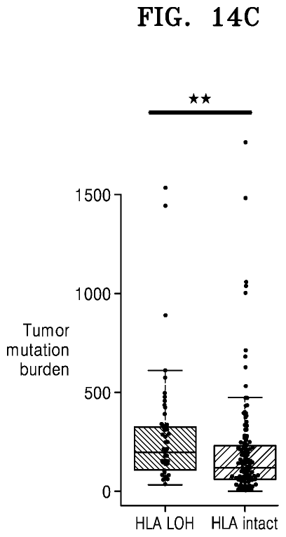

FIGS. 13A and 13B are graphs showing PFS or EGFR mutations by TP53 in the never-smoker group treated with ICI.

Referring to FIGS. 13A and 13B, it was confirmed that TP53 and EGFR mutations were important biomarkers in never-smokers treated with ICIs. In addition, it was confirmed that the TP53 mutations were associated with decreased responses to ICIs in never-smokers.

3.5 Correlation Between Loss of Heterozygosity (LOH) and TMB in Class 1 HLA

To investigate the effect of the loss of heterozygosity (LOH) locus on TMB and the response to ICIs in class 1 human leukocyte antigen (HLA), HLA LOH frequencies were observed in 198 patients with non-small cell lung cancer.

In detail, the HLA LOH frequencies were observed in 198 non-small cell lung cancer patients using a highly sensitive LOH detecting pipeline for accurately calculating a specific copy number of the HLA locus. The present inventors identified 54 out of 198 patients with LOH in at least one HLA-I locus. That is, 27% tumors with LOH were observed in at least one HLA-I locus.

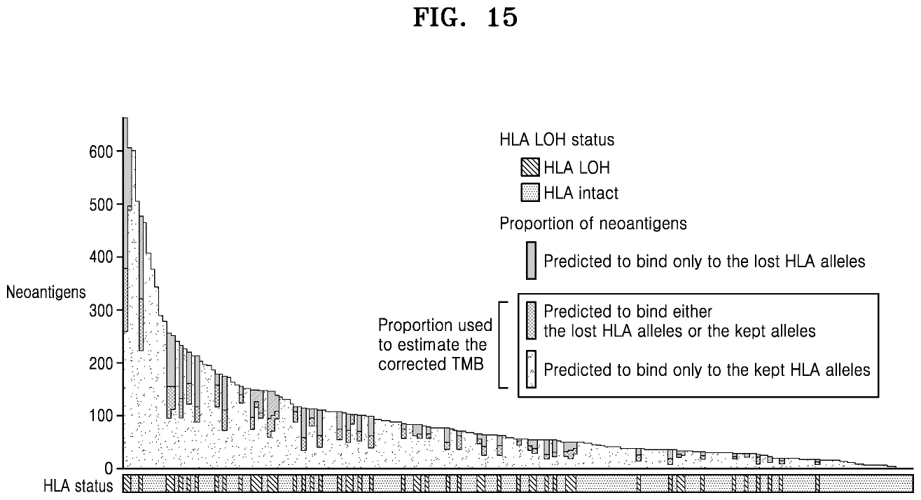

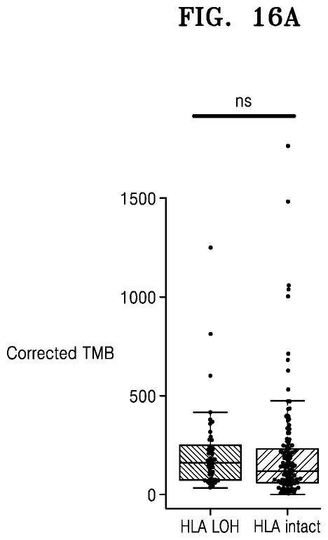

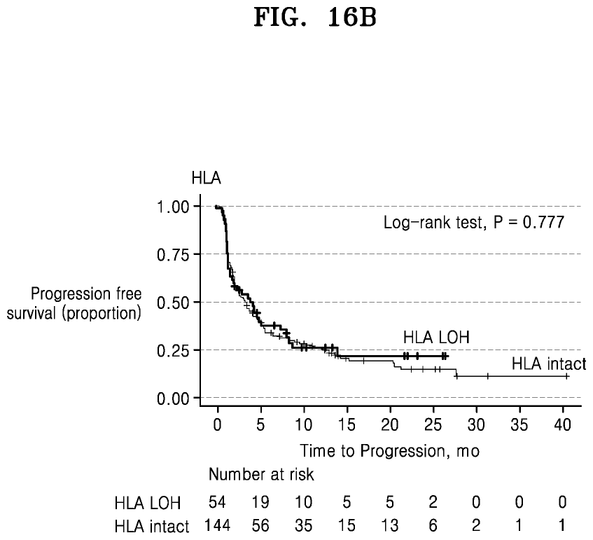

FIG. 14A shows frequencies of HLA LOH events in patient groups.

FIG. 14B shows an association between HLA intact and HLA LOH in patient groups.

FIG. 14C shows a comparison of the HLA intact and the HLA LOH, which is not related to the elevated ORR, but is related to the corrected TMB.

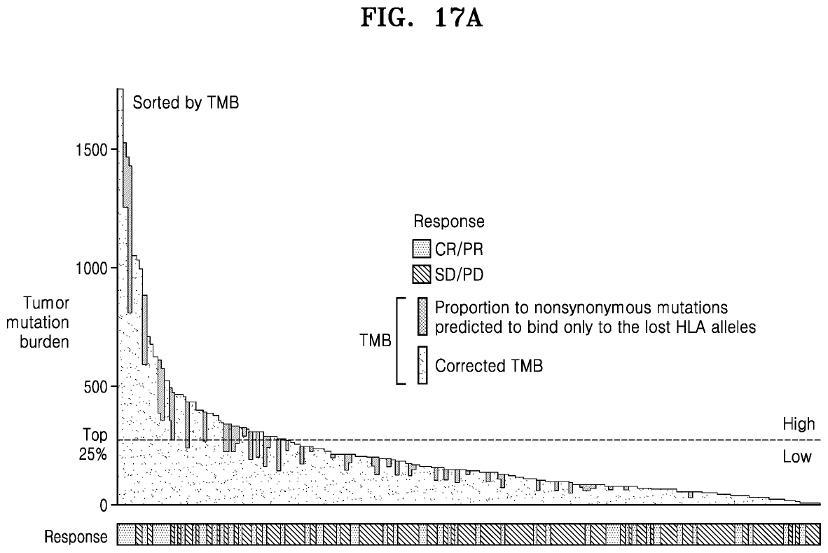

FIG. 15 shows neoantigens for each patient, predicted to bind to the lost HLA LOH or the kept HLA LOH.

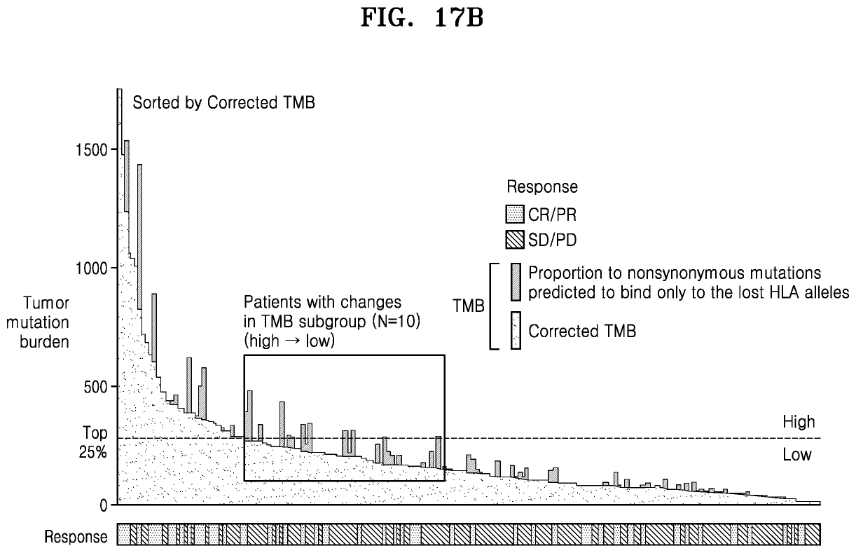

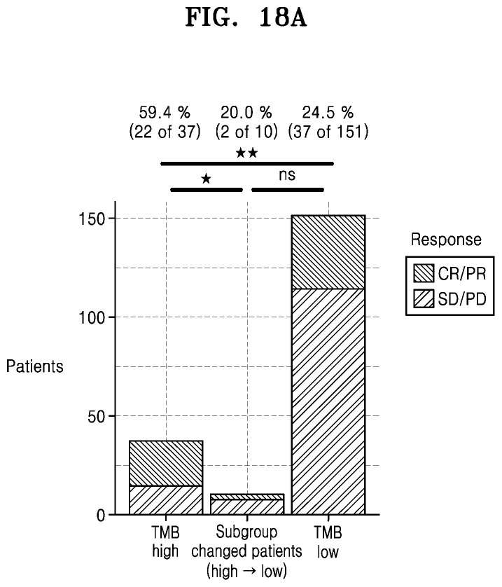

FIG. 16A shows an association between corrected TMB and HLA LOH in patient groups.

FIG. 16B shows PFS in patient groups classified by HLA LOH status.

Referring to FIGS. 14 to 16, there was no association between HLA LOH and an improved response to an anti-PD-L1 agent.

As described above, since the association between HLA LOH and the ICI responses to TMB or a high mutation burden has not been found, the present inventors investigated whether a high mutation load in LOH patients was simply caused by binding to neoantigens irrelevant to HLA alleles and had little to do with an anti-tumor immune response. First, as shown in FIG. 11, the proportion of neoantigens predicted to bind to HLA alleles and the corrected TMB were computationally designed.

As a result, the corrected TMB was not calculated such that the patients with HLA LOH had a higher TMB than the controls. This indicates that HLA LOH proposes immune-editing and has an impact on the increase of TMB. This finding suggests that existing TMB calculating methods are inadequate to some patients having an inhibitory antigen presentation pathway.

3.6 Association Between Corrected TMB and Response to ICIs in HLA

Given the association of corrected TMB with antigen presentation in HLA genes, the present inventors investigated whether the corrected TMB had another advantage beyond the conventional TMB.

First, the corrected TMB was applied to HLA LOH samples to classify the patients into a high TMB group and a low TMB group.

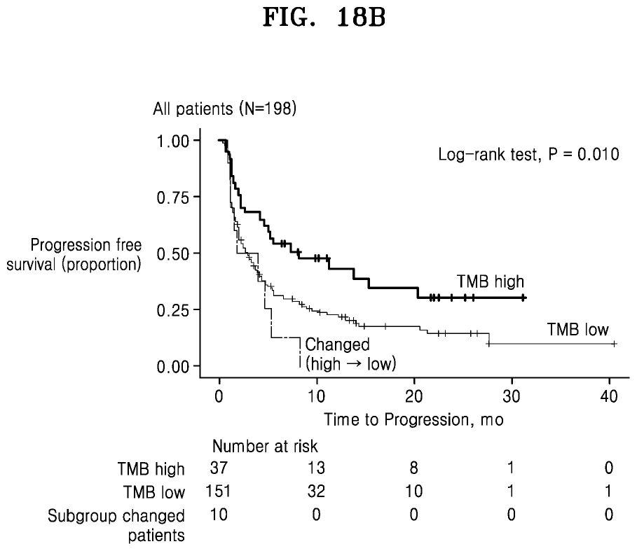

FIGS. 17A and 17B show proportions of nonsynonymous mutations binding only to the lost HLA alleles and only to the kept alleles.

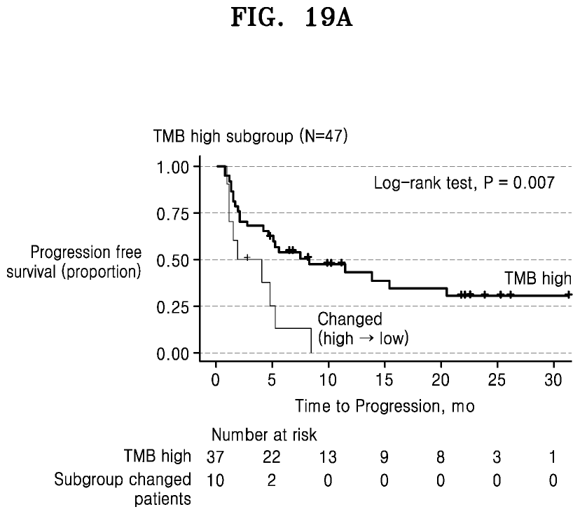

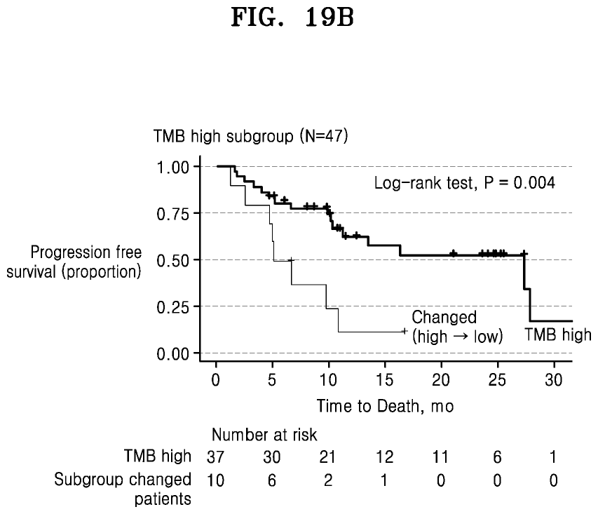

FIG. 18A shows a distribution of patient groups changed in subgroup by corrected TMB from a high TMB group to a low TMB group.

FIG. 18B shows relapse or death rates of patient groups changed in subgroup by corrected TMB from a high TMB group to a low TMB group.

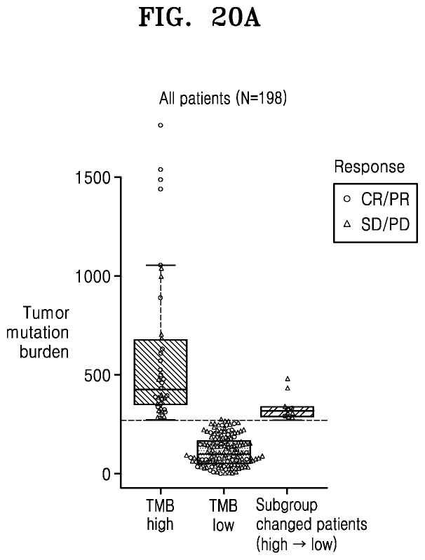

FIGS. 19A and 19B show relapse or death rates of patient groups reclassified as a high TMB group by corrected TMB.

As shown in FIGS. 17 to 19, it was confirmed that ten patients were reclassified from the high TMB group to the low TMB group. Specifically, it was confirmed that the objective response rate (ORR), PFS and OS were all low in the patients with changes in subgroup from the high TMB group to the low TMB group. This data means that HLA LOH is associated with decreased antigenicity of tumors (n=9, median TMB was 308 mutations) and has an impact on the response to ICIs (n=10, TMB (range: 282-477 mutations) versus corrected TMB (range: 143-271 mutations)).

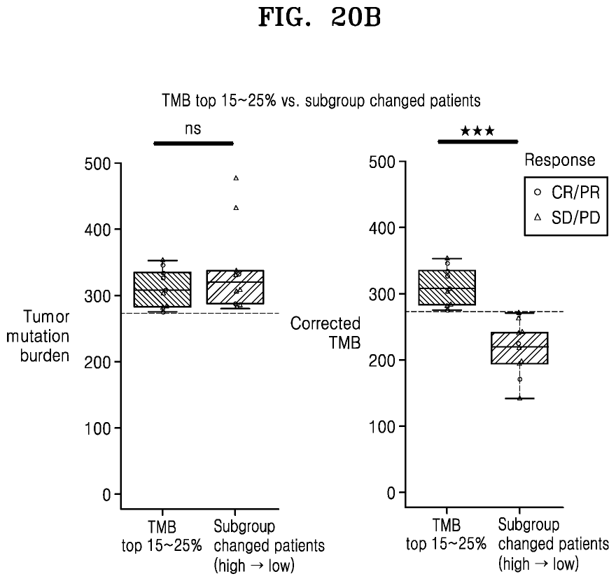

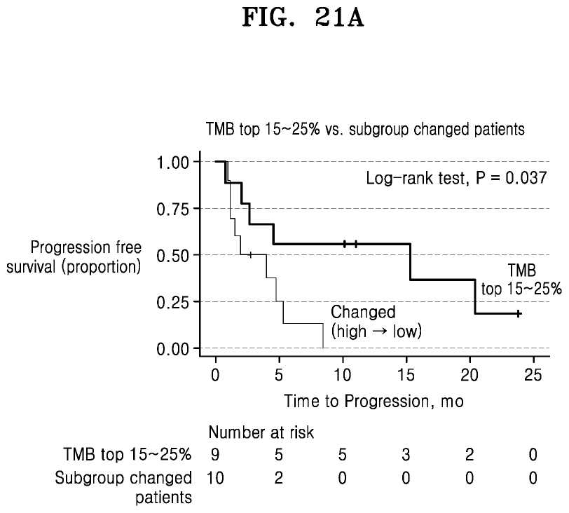

FIG. 20A shows a distribution of TMB values in the whole patent cohort by corrected TMB.

FIG. 20B shows a distribution of TMB values in subgroup changed patient cohorts by corrected TMB.

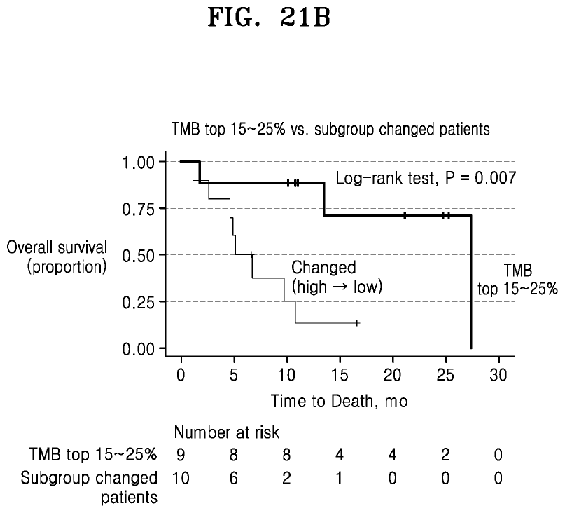

FIGS. 21A and 21B show time-dependent relapse or death risks of subgroup changed patient cohorts by corrected TMB.

As shown in FIGS. 20 and 21, despite similar TMB values, the subgroup changed patients had lower PFS and OS than the top 15-25%.

Next, the present inventors investigated whether the corrected TMB had additional estimates for discriminating responders from non-responders by comparing top 37 patients with corrected TMB (corrected TMB≥272 mutations) with top 37 patient with TMB.

The result confirmed that the corrected TMB was better than the conventional TMB in predicting PFS (for corrected TMB high versus low, PFS log-rank p=0.005, and for TMB high versus low, PFS log-rank p=0.020, data not shown).

3.7 Clinical Usability of TMB-PD-L1 Combination

To validate the clinical usability of a combination of TMB and PD-L1, PD-L1 expression was identified in 144 patients, and it was confirmed that the patients with high PD-L1 expression levels (≥50%) produced 42% of ORR. The PD-L1 expression was significantly high in OR patients (CR/PR versus SD/PD, Mann-Whitney test, P=0.0020). The TMB and the PD-L1 expression could objectively discriminate the response group from non-response group in a similar extent (PD-L1 AUC=0.66, 95% CI, 0.56-0.76, data not shown). However, like the TMB, the PD-L1 expression alone was not sufficient to predict the clinical response. The distribution of the PD-L1 expression was similar to that in both of the top and bottom TMB groups (Mann-Whitney test, P=0.1222, the right plot of FIG. 6A), which means that the PD-L1 expression level is not associated with TMB.

Therefore, the present inventors investigated whether the usability as a biomarker was improved when the PD-L1 expression was considered in combination with TMB.

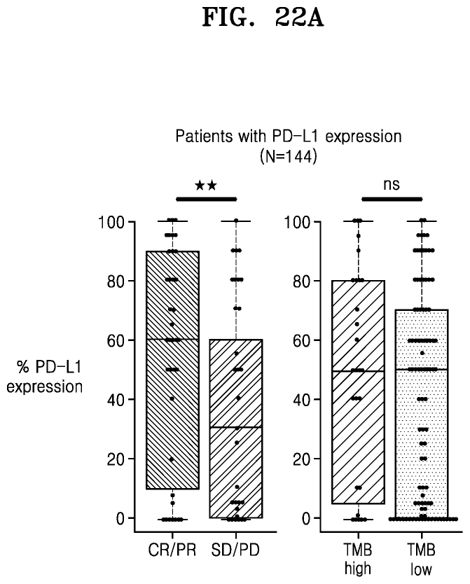

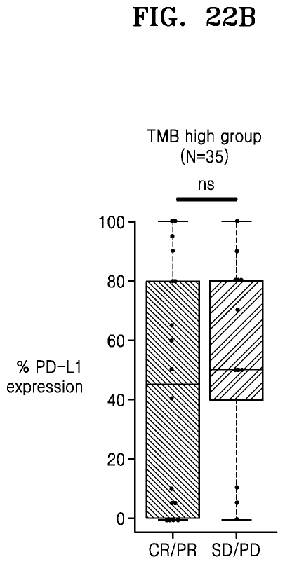

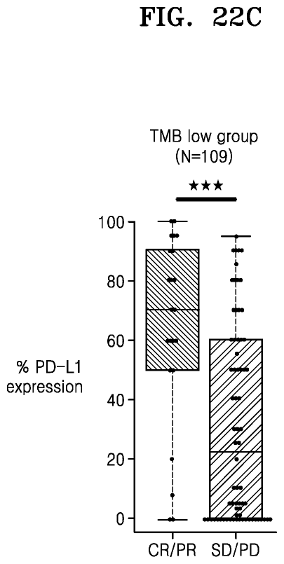

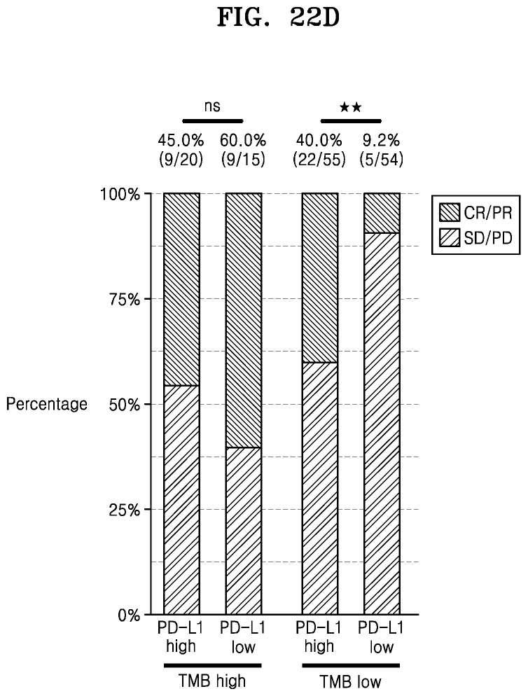

FIG. 22A shows distributions of PD-L1 expression of CR/PR (n=45) versus SD/PD (n=99) patients and PD-L1 expression of high TMB (n=35) versus low TMB (n) patients.

FIG. 22B shows responses to ICIs in TMB high group.

FIG. 22C shows responses to ICIs in TMB low group.

FIG. 22D shows percentages of responses in patient groups classified by TMB and PD-L1.

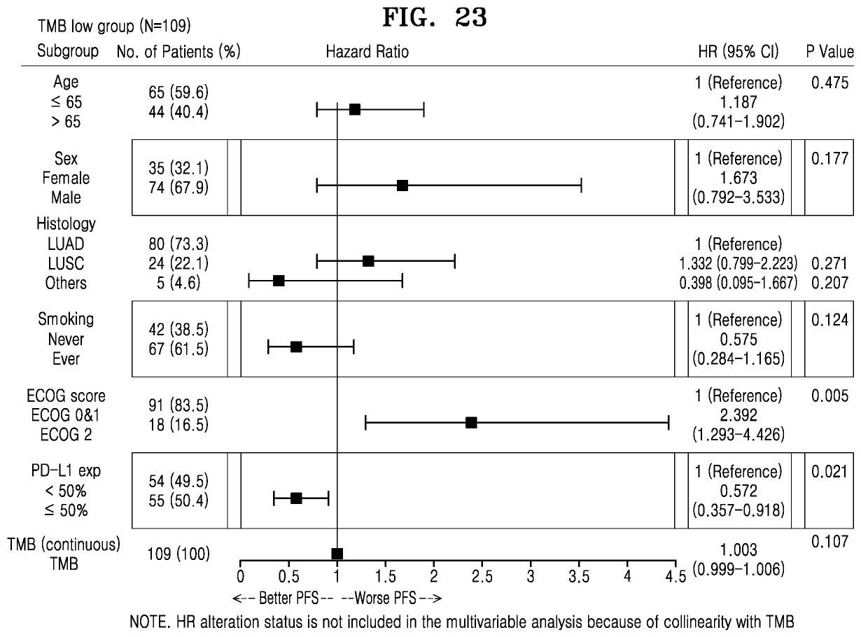

FIG. 23 shows Forest plots representing multivariate cox proportional hazards model.

As a result, as shown in FIGS. 22 and 23, the ORM in the low TMB group was significantly higher in the high PD-L1 expression group than in the low PD-L1 expression group (<50%) (P=0.0003), and the ORR in the high TMB group was similar in both groups.

Taken together, this data means that the PD-L1 expression is more highly predictive in the low TMB group than in the high TMB group (for the low TMB group, PD-L1 AUC=0.75, 95% CI, 0.64-0.86, and for the high TMB group, PD-L1 AUC=0.56, 95% CI, 0.36-0.76, data not shown).

In more detail, the progression or death risk rates were 0.58 in progression free survival (PFS) (95% CI, 0.38-0.89, P=0.014) and 0.55 in overall survival (OS) (95% CI, 0.31-0.95, P=0.033), corresponding to high PD-L1 expression versus low PD-L1 expression group death rates, respectively, in the low TMB group. The multivariate analysis confirmed that the PD-L1 expression was independently associated with PFS (HR=0.57, 95% CI, 0.36-0.92, P=0.021) and OS (HR=0.51, 95% CI, 0.27-0.97, P=0.039) in the case of controlling variables for age, sex, morphology, smoking status, or ECOG score in the low TMB group.

To validate the results, 75 tumors in the independent ICI-treated group (resourced ICI-cohort) were further analyzed. The PD-L1 expression was assessed with 70 patients out of 75 patients, and ten patients out of the assessed 70 patients showed a high PD-L1 expression level (≥50% expression).

### Example 4

### Identification of Biomarker Predictive of Response to ICIs

4.1 Identification of Limitations and Potentials of PD-L and TMB

As described above, the present inventors carried out a comprehensive genomic analysis of advanced NSCLC samples to investigate the role of genomic function in determining the response to ICIs, and could find out limitations of TMB and PD-L1 expression as biomarkers as well as the results consistent with the findings of the previous studies. In addition, it was observed that the genetic features associated with cancer immunotherapy, including HLA LOH, HR deficiency and TP53 mutations in never-smokers, could impact the response to ICIs.

In the above-described Examples, it was observed that a low TMB patient showed a significantly positive correlation with PD-L1 expression and response to ICIs, whereas a high TMB patient showed a good response to ICIs irrespective of PD-L1 expression. In addition, it was confirmed that use of a high TMB cutoff value (top 20-25%) was helpful in defining a “high TMB”, and it was also assessed that combining PD-L1 with TMB was beneficial to clinical usability. In conclusion, it was confirmed that PD-L1 provided improved clinical utility when it is applied in combination with TMB.

4.2 Identification of Association Between Response to ICIs and HLA LOH

In Example 3, the present inventors found out that HLA LOH was abundant in NSCLC before immunotherapy (27%) and was not associated with the decreased response to an antibody targeting PD-1/PD-L1. Such discrepancy may possibly be accounted for by colligated tumor types of patients of the existing study cohorts or treatment of the patients by anti-CTLA-4, anti-PD-1 or a combination therapy thereof, rather than by anti-PD1 monotherapy. This finding suggests that MHC class I expression is not associated with primary resistance to anti-PD-1 agents among patients with melanoma.

Because of the unexpected discrepancy between the observed results and immune surveillance hypothesis, the present inventors investigated whether the association of HLA-LOH and response to ICIs would be more accurately characterized by considering a tumor mutation burden (TMB).

As a result, tumors with HLA-LOH showed a higher TMB than tumors without HLA-LOH, whereas the HLA-corrected TMB was not significantly increased in tumors with HLA-LOH. This observation supports the conceptual hypothesis that HLA-LOH allows for subsequent subclonal expansion 18. The increased TMB was identified in tumor samples exhibiting HLA-LOH, and it was confirmed that the increased TMB would contribute to subclonal neoantigens predicted to bind to the lost HLA locus. Such data suggests that the neoantigens predicted to bind to the lost HLA alleles may contribute to subclonal expansion, which leads to the increase in the TMB of tumors representing a loss of HLA.

In addition, it was confirmed that the prediction accuracy was increased by introducing the corrected TMB. More specifically, the approach of taking the HLA LOH into account made it possible to adjust an mismatch value observed from the relationship between the high TMB and the increase to ICIs. As a result, the present inventors established a hypothesis that effective cancer immunotherapy may not be derived by the neoantigens predicted to bind only to the lost HLA alleles. Such a result means that the corrected TMB is more advantageous in discriminating patients not responding to ICIs despite of a high TMB.

4.3 Identification of Association Between HR Gene Alteration and TMB

In the above-described Examples, the present inventors analyzed that the HR gene alteration is associated with higher TMB and longer PFS. Cytosolic DNA fragments derived from defective DNA damage response and repair mechanisms may influence the response to ICIs by triggering the stimulator of interferon genes (STING) signaling pathway, which may exert additional influences on DNA repair alterations in the immune checkpoint inhibitor (ICI) response. Various cytoplasmic DNA sensors are assumed to bind cytosolic DNA fragments and to activate the STING pathway, which induces antitumor activity via type 1 interferon and T cell recruitment. In addition, it was observed that STK11 loss leads to immune evasion through methylation-induced STING suppression. Actually, STK11-mutation was associated with primary resistance to ICIs in NSCLC patient cohorts. It was also observed that ¾ (75%) patients having STK11 truncated mutations did not reach objective responses. As a whole, these studies imply clinical usability of the STING pathway in immunotherapy. For example, increasing dependent anti-tumor activities may become an effective combination strategy in patients not responding to ICIs.

4.4 Identification of Prediction Result of Response to ICIs in Never-Smoker Non-Small Cell Lung Cancer Patients

In the study cohort of this experiment for confirming whether a non-small cell lung cancer (NSCLC) occurring to a never-smoker has a different action mechanism from a smoker NSCLC due to a mutation inducing effect of a carcinogen, it was found that PD-L1 expression and TMB were not sufficient to determine the response to ICIs in smoker patients. This finding suggests that essential somatic mutations are important in determining the response to ICIs. In addition, it was confirmed that a patient with EGFR mutation could not show an objective response. It was also confirmed that the TP53 mutation was associated with a decreased response to ICIs despite known associations with increased TMB and PD-L1 expression. In preclinical models, initial data imply that production of chemokine having a reduced loss of p53 function reduces immune cell infiltration. Indeed, immune escapes of tumors were achieved by inactivating anti-tumor priming and tracking periods.

The results suggest that a corrected TMB is a more reliable biomarker than a conventional TMB, specifically in the never-smoker patient cohort. In addition, additional genetic features, such as HR gene alteration or STING pathway, may contribute to understanding and prediction of a response to ICIs.

1. A method for analyzing a corrected TMB, comprising the steps of:
sequencing a biological sample obtained from a cancer patient;
filtering data output from the sequencing;
calculating a tumor mutation burden (TMB) using the filtered sequencing data; and
correcting the calculated TMB using Equation 1:
$$Corrected TMB = TMB \times \frac{( NeoAg - NeoAgL + NeoAgC )}{NeoAg} \qquad [ Equation 1 ]$$

2. The method of claim 1, wherein the cancer patient is a lung cancer patient.

3. The method of claim 1, wherein the cancer patient is a never-smoker.

4. A method for providing information for predicting a response to cancer immunotherapy in a cancer patient, the method comprising the step of:
sequencing a biological sample obtained from a cancer patient;
filtering data output from the sequencing;
calculating a tumor mutation burden (TMB) using the filtered sequencing data;
correcting the calculated TMB using Equation 1 below; and
providing information for predicting the response to cancer immunotherapy in the cancer patient with the calculated TMB:
$$Corrected TMB = TMB \times \frac{( NeoAg - NeoAgL + NeoAgC )}{NeoAg} . \qquad [ Equation 1 ]$$

5. The method of claim 4, wherein the predicting of the response includes predicting the response to cancer immunotherapy in the cancer patient to be high when the corrected TMB value is top 20 to 30% or higher, and to be low when the corrected TMB value is bottom 20 to 30%, as compared with the whole patient data.

6. The method of claim 4, further comprising:
measuring an expression level of programmed death-ligand 1 (PD-L1) from the biological sample obtained from the cancer patient; and
predicting the response to cancer immunotherapy by combining the measured expressed level of PD-L1 with the corrected TMB value.

7. The method of claim 4, wherein the cancer immunotherapy is an immune checkpoint inhibitor (ICI).

8. The method of claim 7, wherein the immune checkpoint inhibitor (ICI) is anti-PD-L1, anti-PD-L1, anti-PD-1, or anti-CTLA-4.

9. The method of claim 4, wherein the cancer of the cancer patient is selected from the group consisting of lung cancer, melanoma, Hodgkin lymphoma, stomach cancer, urothelial cell cancer, head and neck cancer, liver cancer, colon cancer, prostate cancer, pancreatic cancer, liver cancer, testicular cancer, an ovarian cancer, endometrial cancer, cervical cancer, bladder cancer, brain cancer, breast cancer, and kidney cancer.

10. The method of claim 9, wherein the lung cancer is a non-small cell lung cancer or a small cell lung cancer.

11. The method of claim 4, wherein the cancer patient is a never-smoker.

12. A computer-readable recording medium having a program recorded therein for executing the method of claim 1 on a computer.

13. An apparatus for analyzing a response to cancer immunotherapy in a cancer patient, the apparatus comprising:
a data generation unit for generating a set of gene data by performing sequencing on a biological sample obtained from a cancer patient;
a calculation unit for calculating a TMB by performing filtering the generated genetic data; and
a correction unit for calculating the calculated TMB into a corrected TMB value using Equation 1:
$$Corrected TMB = TMB \times \frac{( NeoAg - NeoAgL + NeoAgC )}{NeoAg} . \qquad [ Equation 1 ]$$

14. The apparatus of claim 13, further comprising an analysis unit for predicting the response to cancer immunotherapy in the cancer patient to be high when the corrected TMB value is top 30 to 0%, and to be low when the corrected TMB value is bottom 30 to 0%, as compared with the whole patient data.

15. The apparatus of claim 13, further comprising:
a measuring unit for measuring a PD-L1 expression level from the biological sample obtained from the cancer patient; and
a determination unit for determining a response to cancer immunotherapy by combining the measured PD-L1 expression level with the corrected TMB value.

16. The apparatus of claim 13, wherein the cancer of the cancer patient is selected from the group consisting of lung cancer, melanoma, Hodgkin lymphoma, stomach cancer, urothelial cell cancer, head and neck cancer, liver cancer, colon cancer, prostate cancer, pancreatic cancer, liver cancer, testicular cancer, an ovarian cancer, endometrial cancer, cervical cancer, bladder cancer, brain cancer, breast cancer, and kidney cancer.

17. The apparatus of claim 16, wherein the lung cancer is a non-small cell lung cancer or a small cell lung cancer.

18. The apparatus of claim 13, wherein the cancer patient is a never-smoker.

19. A computer-readable recording medium having a program recorded therein for executing the method of claim 4 on a computer.
